(19) United States
(12) Patent Application Publication
(10) Pub. No.: US 2025/0241723 A1
(43) Pub. Date: Jul. 31, 2025

(54) RING-HANDLED INSTRUMENT STRINGER RACK AND METHOD

(71) Applicant: Case Medical, Inc., Bloomfield, NJ (US)

(72) Inventors: Marcia A. Frieze, Montclair, NJ (US); Shirel Navon Ravinay, Dumont, NJ (US)

(73) Assignee: Case Medical, Inc., Bloomfield, NJ (US)

(21) Appl. No.: 19/170,940

(22) Filed: Apr. 4, 2025

Related U.S. Application Data
(63) Continuation-in-part of application No. 18/201,905, filed on May 25, 2023.

(51) Int. Cl. A61B 50/22

(52) U.S. Cl. CPC A61B 50/22

(57) ABSTRACT

A rack and “U-shaped” stringers for use in stringing of ring-handled medical instruments in preparation for at least one of an assembly operation, an assembly count operation, a storage procedure, a decontamination operation, a sterilization process, and/or a disinfection operation, involving such instruments.

### PRIORITY CHAIN

This application is a Continuation-In-Part of U.S. patent application Ser. No. 18/201,905, filed 2023 May 25 which was published as US Publication Application No. US2024/0390093 on 2024 Nov. 28.

### FIELD OF THE INVENTION

The present invention relates to the field of devices for organizing ring-handled medical instruments for a variety of purposes, especially for sterilization thereof. It further relates to an improved method of organizing and assembling such ring-handled medical instruments for subsequent sterilization or disinfection thereof in preparation for a medical or surgical procedure, as compared to prior methods. It further relates to racks and “U-shaped” stringers for use in stringing of ring-handled medical instruments in preparation for at least one of an assembly operation, an assembly count operation, a storage procedure, a decontamination operation, a sterilization process, and/or a disinfection operation, involving such ring-handled instruments. In some embodiments, it optionally further includes the use of a “driver tool” for ease of maintaining an “open position” of the instruments, in particular by easing the prevention of the closing of a “box lock” (typically present on many such instruments) during the stringing operation.

### BACKGROUND OF THE INVENTION

Instrument racks and trays are well known in the art. Ring handled instruments are commonly used in most surgical procedures. For sterilization and decontamination, it is critical for the instrument tips to be in an open position. Some facilities use a rolled towel in the operating room to keep these instruments open and to facilitate the instrument count procedure. Nonetheless, this is cumbersome as the towel does not keep the instruments grouped in place. Further, the towel must be laundered and can cause lint to adhere to the surgical field. Others use an instrument stringer to group the instruments to ensure that the devices remain open during sterilization. A small representative sample is illustrated by U.S. Pat. No. 5,137,151 (which has a support for gripping the instrument, a rod that goes through one ring of one part of the instrument, and a second support that a second ring of the instrument (or a portion close thereto) rests upon); U.S. Pat. No. 4,229,420 has a wall which one ringed handle goes behind and a rear angled portion is pushed inward to force the second ringed handle to separate from the first; U.S. Pat. No. 6,230,888 has a cylindrical portion which when forced against the pair of ringed handles of the instruments forces the ringed handles apart; U.S. Pat. No. 9,259,272 has independent rods strung through a first ring of a series of ring handled instruments and a second rod strung through a second ring of that series of instruments and the two rods are then assembled together with a spacer to assure that the instruments are in the open position for sterilization purposes; and US 2005/0040066 shows a grooved, angled tray for ring-handled instruments and configured to make it easier for medical personnel to grab the ring-handled item for use in the course of performing a medical procedure. Each of these US patents and US Published patent applications are incorporated in their entirety herein by reference, but only to the extent that they supplement and do not contradict the express statements made in this specification. In the event of any contradiction between this specification and one or more of the above references, the statements in this specification shall prevail.

Each of the inventions in the forgoing patents suffers from at least one of the following: (a) they are cumbersome to use, (b) they require multiple hands to string the instruments, (c) they are overly heavy; or (d) they do not relate to readying the ring-handled instruments for sterilization, among other problems. An instrument stringer as a device does not facilitate the instrument count procedure. The instrument count is a required procedure for best practices to ensure that the instruments selected are assembled per procedure. Generally, a count sheet or bill of materials is available to list the instruments required and have a count or quantity of what has been used and needs to be accounted for in the medical procedure.

U-shaped stringers are commonly used to secure ring-handled instruments as a group in the open position, especially for decontamination and sterilization. A variety of scissors, forceps, hemostats, among other instruments having a variety of lengths are needed for most surgical procedures and many non-surgical medical procedures. Many such instruments are used during a procedure and duplicate extras are needed in case of damage or for subsequent use during the same procedure or surgery. Assembling a stringer is very difficult as the rings must be open to avoid box locks closing. Generally, two hands are needed, one to string the instrument, and a second hand to position the instrument. Not only is this awkward, but often the position is not held in place and rings missed during the assembly procedure requires the operator to start over again.

Generally, U-shaped stringers are supplied in pairs, and have either a rounded portion with two upward extending arms extending therefrom when viewed from one particular orientation or the U-shaped stringers may also have the rounded portion replaced by a substantially flat crossbar connecting the two arms resulting in a U-shaped stringer which might best be described as a rectangular U-shape or alternatively resembling a “staple” shape (both of which will be referred to hereinafter as “U-shaped”. The arms of either of these are inserted through the rings of the ring-handled instruments to group the so strung instruments into a group assembly. In a modified design of the U-shaped stringer, it may further have may have a lock or hinged closure, which hinged closure links the free ends of the U-shaped stringer arms. In these alternate stringers, a first end of the cross bar may extend from a hinge at the free end of one of the arms of the U-shaped stringer and have a second end capable of engaging in a locking relationship with the free end of the second arm of the U-shaped stringer. A second alternative U-shaped stringer has a cross bar extending from a hinge at each of the free arms of the U-shaped stringer in which both of such cross bars can rotate so as to meet in the distance between the two free arms and engage with one another in a locking relationship with each other. These stringer devices still remain awkward to string and the hinge or locking mechanism frequently breaks or sticks in place during the assembly or stringing process. There are other devices designed for other purposes like an instrument count rack. This is intended, as the name implies, to keep track of the count of the various instruments so that there is no instrument that is unaccounted for which might have been left inadvertently within a patient. However, such other devices are heavy, complicated, hard to manufacture, and take up too much space within a surgical tray. Another design is simply a storage rack, which cannot be used for stringing or assembling such devices as the design inhibits the insertion process required for sterilization or decontamination. This is because the sides and base of this storage rack occlude the instrument surface; and further the tips of the instruments are closed; further, long instruments placed in a downward slope will hit the table surface or slide forward; and still further the stringer cannot be inserted into the rings of the handles of such instruments when in such storage racks, primarily due to intervening side wall portions. Further, due to the side walls not having openings, placement of the instruments in them for assembly purposes, specifically in stringing assembly processes, is not possible.

### OBJECTS OF THE INVENTION

An object of some embodiments of the invention is to provide a product which allows for a simple, lightweight, easy to use device and method for stringing, assembly of ring-handled medical instruments in preparing them for sterilization/decontamination and preparation for a medical procedure, or for any reason that sterilization may be required.

Another object of some embodiments of the invention is to provide a relatively simple stringer method for ring-handled medical instruments in preparing such instruments for a sterilization/decontamination procedure.

A still further object of some embodiments of the invention is to provide a simple, easy to use stringer rack for ring-handled medical instruments in organizing them for sterilization/decontamination after being used in a medical procedure, or for any reason that sterilization/decontamination may be re-required.

A still further object of some embodiments of the invention is to provide an instrument rack and a stringer for use in combination with one another in the preparation of ring-handled medical instruments for a sterilization/decontamination procedure to be conducted on or a subsequent medical procedure with such medical instruments.

Another embodiment of the invention will allow for the instrument count procedure to commence during the assembly process such that the right amount of ring handled instruments are selected per the count sheet or bill of materials and instruments can be organized and grouped prior to insertion of and assembly with the stringer.

A further embodiment employs a “driver tool” which drives the ringed instrument into an open position and maintains them in the open position during the stringing operation step.

Yet a further embodiment includes “recesses” in the rack top surface for acceptance of the driver tool to further aid in maintaining the ringed instruments in the open position during the stringing process step.

A still further embodiment includes a “channel” in the rack for storage of the driver tools when not in use.

Yet other object of the invention embodiments will be apparent to those of ordinary skill in the art after having benefit of the present disclosure.

### SUMMARY OF THE INVENTION

Some or all of the above objects can be achieved by utilizing one or two U-shaped stringers (inclusive of both curved “U” shaped and rectangular analogs as in an unused “staple”), each having two upraised arms (a first arm and a second arm) when positioned to look like a “U”. Ring-handled medical instruments are positioned in a rack/holder (discussed further below) with the two rings of the ring-handled instruments vertically above one another and separated so that the instruments are in an open position. This allows for the stringer to be inserted (in either direction through the rings (one arm through the rings that are lower than the other rings (i.e., lower rings), and the second arm through the rings that are vertically above the lower rings (i.e., the upper rings)). The second U-shaped stringer (if required) is then inserted from the opposite direction, one arm through the lower rings and one arm through the upper rings. The two U-shaped stringers are then pulled (or pushed) to continue the direction of movement that they went through the various rings to secure the group of medical instruments into a relatively light-weight collection for transport to a suitable tray, containment device, or container for introduction into a decontamination and/or sterilization chamber or for direct introduction into a decontamination/sterilization operation area without such container. Alternatively, a single “U-shaped” stringer having a hinged cross bar and locking mechanism may be used in place of the dual “U-shaped” stringers described above. These alternatives, as described above may have a single, hinged cross bar which can be extending from a free arm of the U-shaped stringer which can be rotated to have a portion at the distal end (from the hinge) of the cross bar received in a receiving area of the other arm of the U-shaped stringer, where it can be locked in place; or each arm of the U-shaped stringer can have a hinged cross bar extending therefrom so that they can rotate into the space between the two arms of the U-shaped stringer, and meet with each other in a locking engagement relationship.

The rack that is utilized in some embodiments with the U-shaped stringers is designed to accommodate a number of ring-handled instruments and can vary in length to accommodate as few or as large a number as may be desired and is only limited by the practicalities of the total quantity and size of the instruments required. For large procedure trays, a second or third rack may be applied adjacent to the first to accommodate the additional instruments and longer stringer that is needed for this grouping. The rack has a flat bottom, a front side and a rear side which are connected to each other via a left side and a right side. The top has top grooves therein for receiving the various shafts/shanks or insertion portion of the ring-handled instruments (other than the ring-handles). The lower ring handles of the instruments sit in a channel that runs between the left side and the right side, perpendicular to the grooves in the top surface, and deeper than those top grooves. This channel has free access to the exterior of the rack through the left side wall and right side wall. The bottom of each top groove is angled when running from the front side toward the rear side so that the bottom of such top groove is highest at the front side to aid in keeping the instruments from sliding forward, and thus helping to keep the ring-handles aligned from one instrument to the next for the stringing/assembly operation.

### BRIEF DESCRIPTIONS OF THE DRAWINGS

FIG. 1 is a perspective view of one embodiment of the rack of the invention.

FIG. 2 is a top view of the rack shown in FIG. 1.

FIG. 3 is a front view of the rack shown in FIG. 1.

FIG. 4 is a rear view of the rack shown in FIG. 1.

FIG. 5 is a left side view of the rack shown in FIG. 1.

FIG. 6 is a right side view of the rack shown in FIG. 1.

FIG. 7 shows a top view of a “U-shaped” stringer, with the free ends of two arms pointed toward the right side.

FIG. 8 shows a perspective view of the “U-shaped” stringer of FIG. 7.

FIG. 9 shows one embodiment of a rack of the invention with sample ring-handled instruments being in place thereon and available for grouping and counting prior to insertion of the “U-shaped” stringer.

FIG. 10 shows one embodiment of a rack of the invention with a variety of sample medical instruments in place with a first “U-shaped” stringer having been strung through the respective rings of the ring-handled instruments.

FIG. 11 shows the same embodiment as in FIG. 10 except that a second “U-shaped” stringer has been inserted through the respective rings of the ring-handled instruments in a direction opposite to the direction of the first “U-shaped” stringer in FIG. 10.

FIG. 12 shows the strung instruments that were seen in FIG. 11, except that the free ends of the two “U-shaped” stringers seen in FIG. 11 have been pulled apart causing the respective connecting portions to be pulled toward each other and clamp down on the exterior two instruments of the group, assuring the respective ends (each distal from the ring-handles) to remain in the open position and can now be transitioned to a decontamination and/or disinfection unit for cleaning and disinfecting of the respective instruments.

FIG. 13 is a top view of an alternate “U-shaped” stringer having a single hinged cross bar.

FIG. 14 is an isometric view of the alternative U-shaped” stringer of FIG. 13.

FIG. 15 is a top view of a second alternative “U-shaped” stringer having two hinged cross bar portions that interact with each other in a locking engagement.

FIG. 16 is an isometric view of the “U-shaped” stringer of FIG. 15.

FIG. 17 is a perspective view of a second embodiment of the invention (similar to that shown in FIG. 1) in which a driver storage channel 30 as well as driver rest grooves 31a, 31b, and 31c are additionally provided.

FIG. 18 is a top view of the embodiment shown in FIG. 17.

FIG. 19 is a left side view of the embodiment shown in FIG. 17.

FIG. 20 is a right side view of the embodiment shown in FIG. 17.

FIG. 21 is comprised of 5 views of one type of driver tool 32 used in connection with the rack shown in FIG. 17; FIG. 21a being a top view, FIG. 21b being a front view (the rear view is a mirror image of the front view and not shown), FIG. 21c being a bottom view, FIG. 21d being a right side view, and FIG. 21e being a left side view of this type of the driver tool 32.

FIG. 22 is a perspective view of the driver tool of the various FIG. 21 views.

FIG. 23 shows a perspective view of an embodiment shown in FIG. 17 loaded with a ring-handled medical instrument with the driver tool 32 in place in the driver tool rest groove 31a.

FIG. 24 shows a perspective view of an embodiment shown in FIG. 17 loaded with multiple varied ring-handled medical instruments with the driver tool 32 completely disposed toward the driver tool rest groove 31a and at rest in such driver rest groove 31a.

FIG. 25 shows four different non-limiting alternative versions of driver tool 32, the alternatives being identified as element 33a, 33b, 33c, and 34d in FIG. 25A, FIG. 15B, FIG. 25C, and FIG. 25D, respectively.

FIG. 26 shows a perspective view of the embodiment of FIG. 17 with two different ring-handled medical instruments (a shorter one in place closer to side 2 and a longer on further from side 2) in place in the stringer rack 1 and two driver tools 32 introduced (a first driver tool 32, with respect to both ring-handled instruments, and a second driver tool 32, with respect only to the longer ring-handled instrument.

### DETAILED DESCRIPTION OF THE INVENTION

Other than when expressly stated as being “critical”, the present specification does not limit the invention to the expressly stated components, shapes, or dimensions thereof, or any of the Figures hereof, but the invention is only limited by the claims. A statement of “importance” shall not be construed as “critical” unless also stated as being “critical”, or as a basis for distinguishing over prior art that cannot be overcome in another manner. All dimensions presented are presented only for exemplification and larger and smaller sizes are acceptable if such larger or smaller sizes (respectively) allow for the rack and stringer system to reasonably operate in the fashion described. The materials of which the rack 1, the “U-shaped” stringers 10, and the driver tools 32 are constructed are presented for exemplification only and are non-limiting unless expressly stated otherwise.

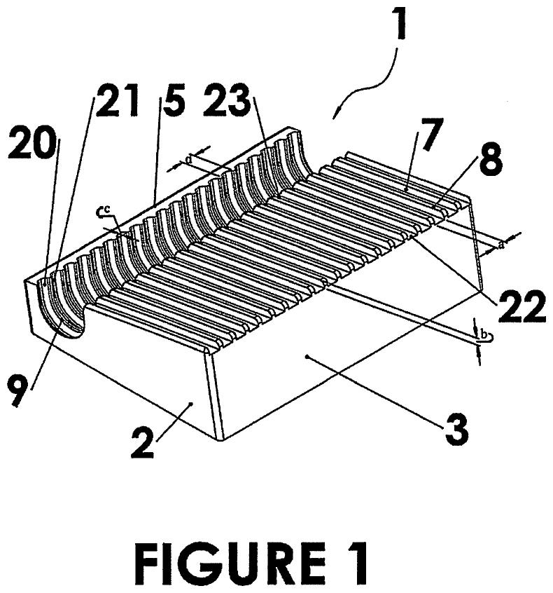

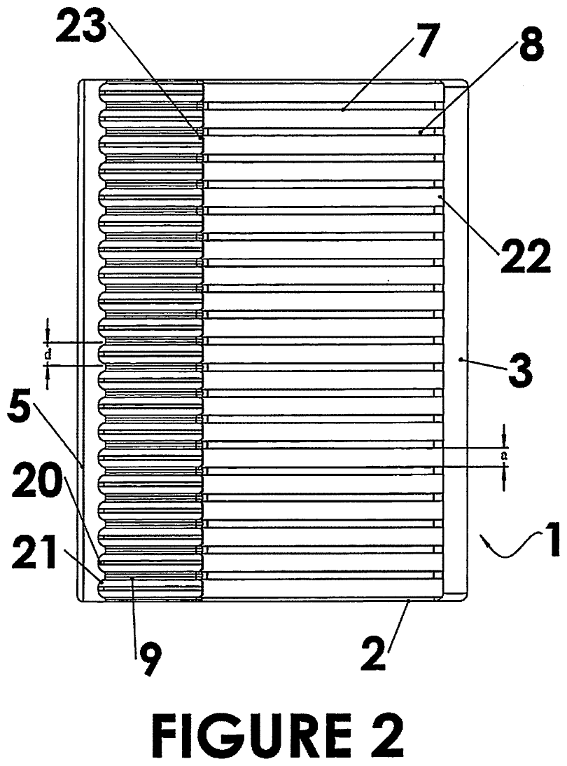

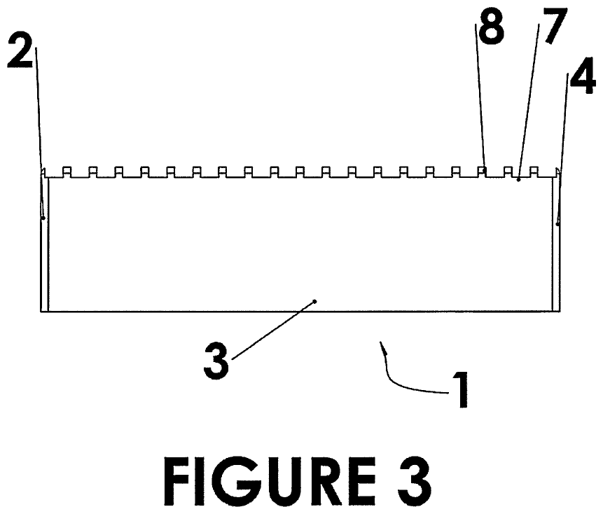

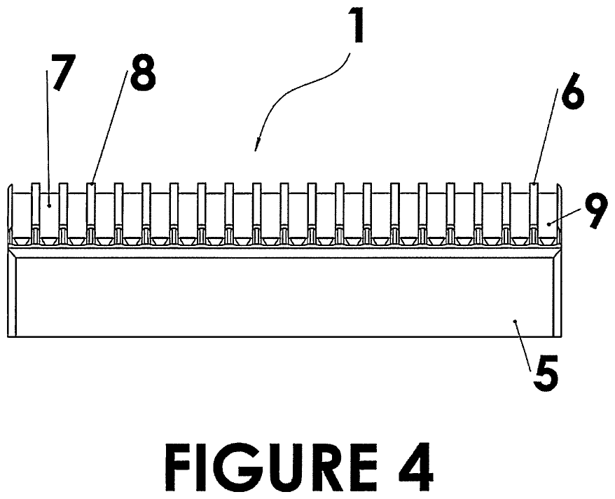

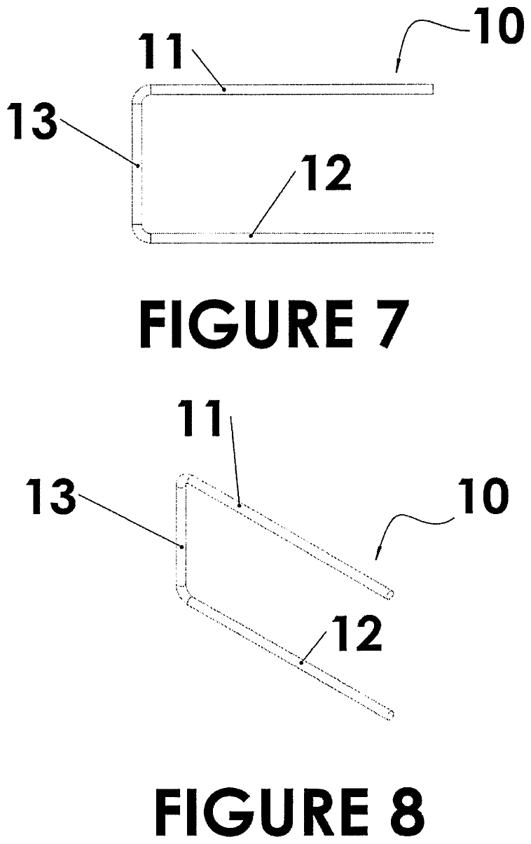

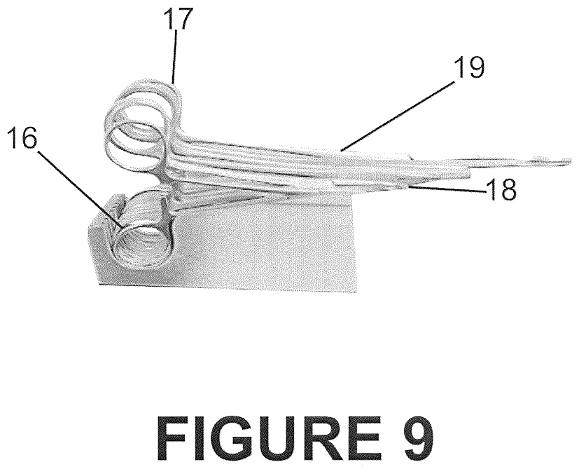

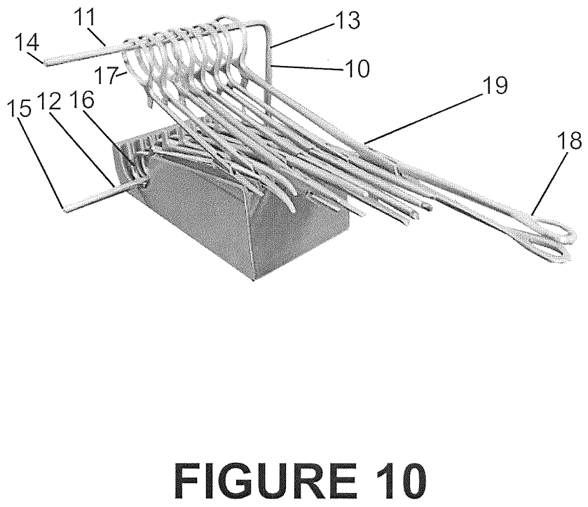

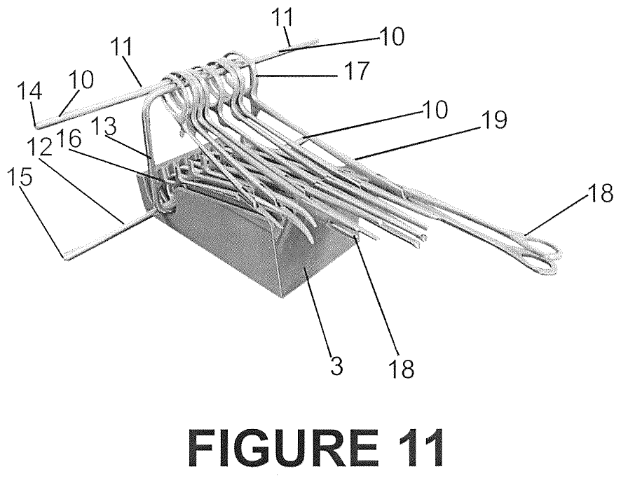

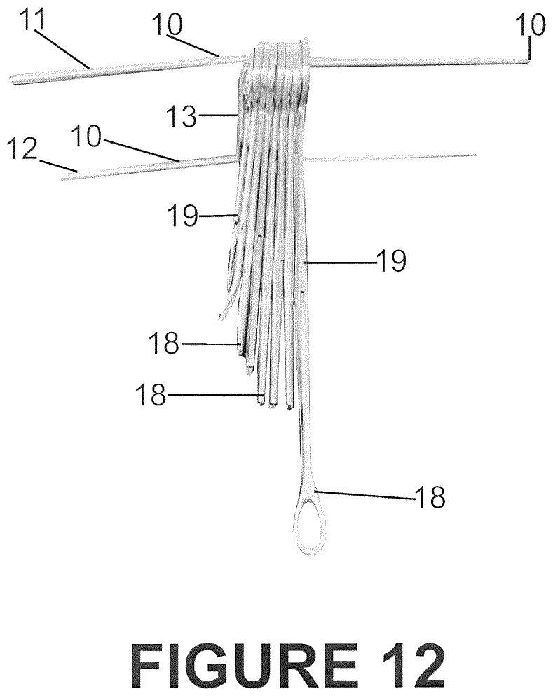

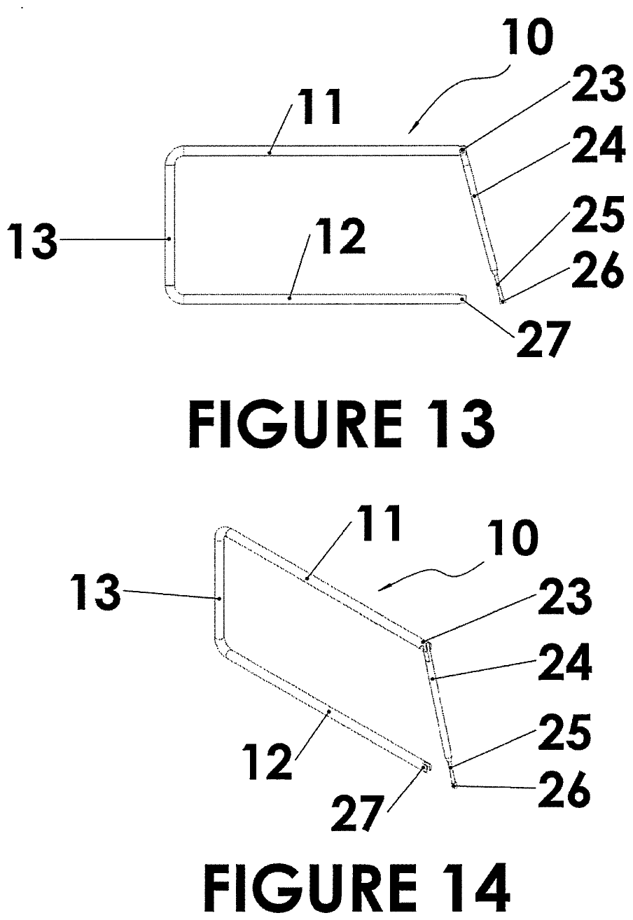

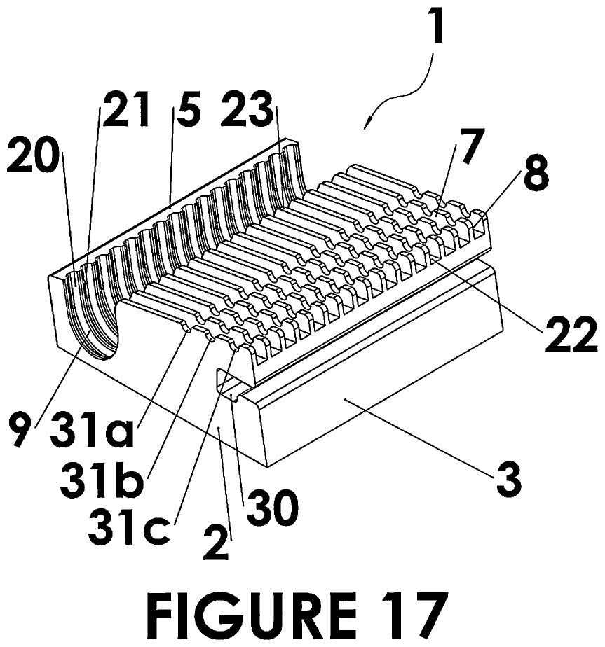

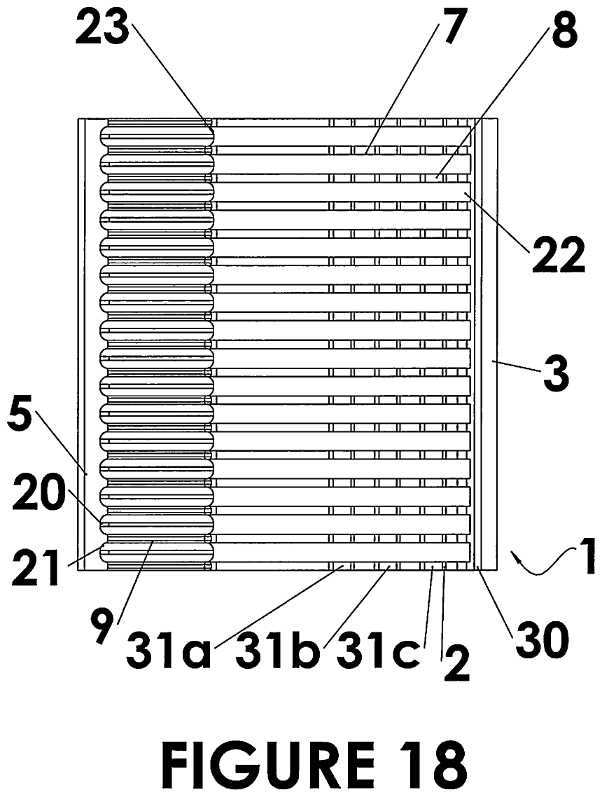

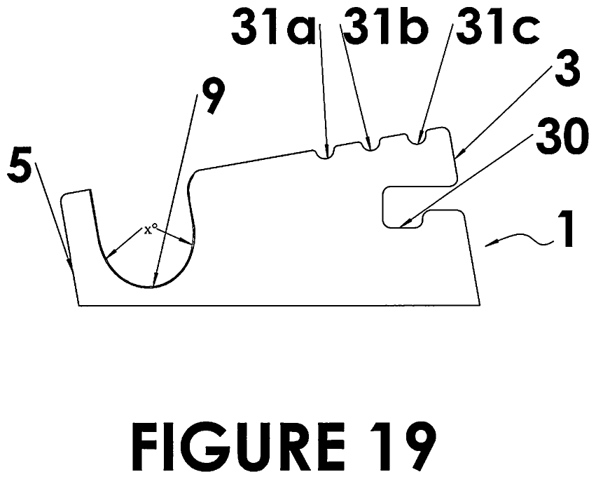

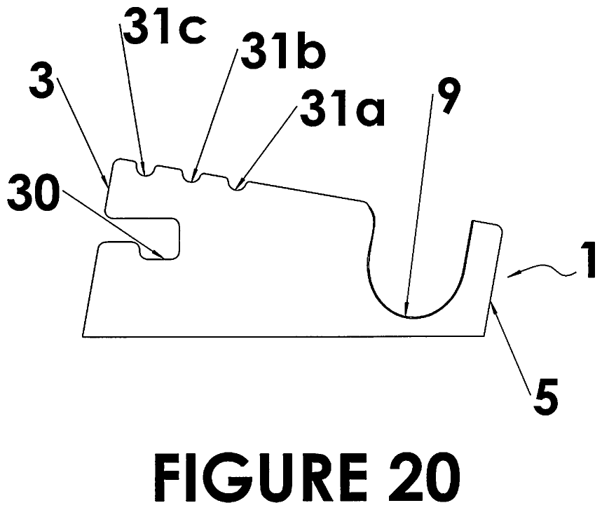

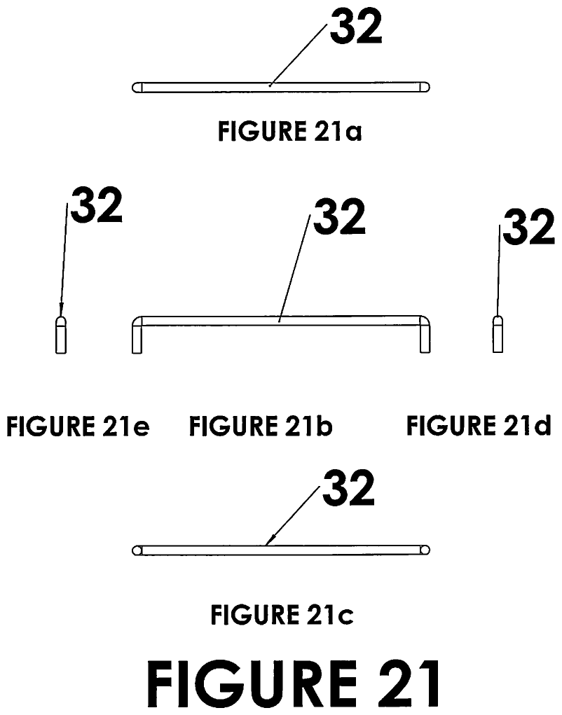

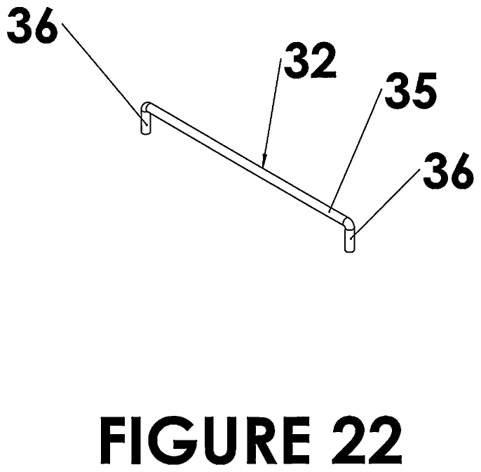

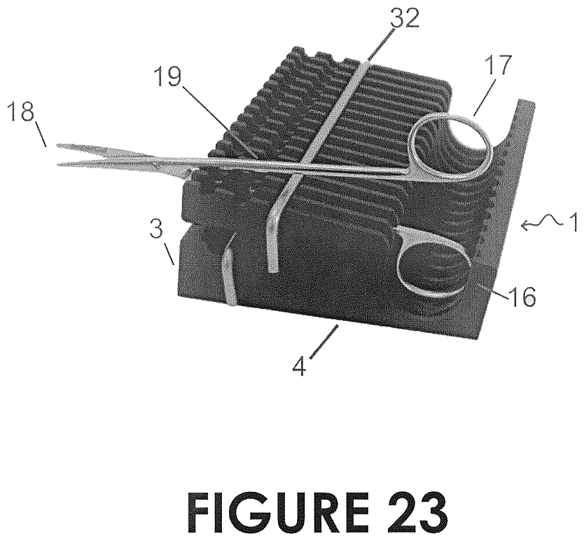

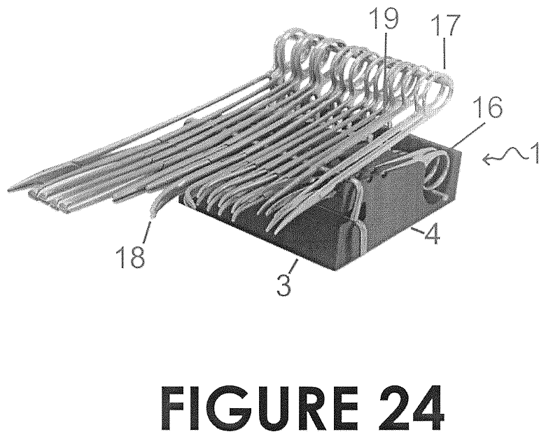

The present invention relates to an instrument rack or storage tray 1 (one non-limiting embodiment of which is shown in the various views of FIGS. 1-6 and a second non-limiting embodiment is shown in FIGS. 17-20) for assembling and securing ring handles instruments and “U-shaped” stringers 10 (one non-limiting embodiment of which is shown in FIGS. 7-8, and exemplary, non-limiting alternative such stringers are shown in FIGS. 13-16). A non-limiting method of use of tray 1 and “U-shaped” stringers 10 (of FIGS. 7-8) are shown in non-limiting general fashion in FIGS. 9-12. FIGS. 21-22 show one embodiment of a driver tool 32 for optional use with the various invention embodiments. FIGS. 23-24 show the use of the driver tool 32 in connection with the ring-handled medical instrument stinger rack of the invention in preparation for insertion of the stringers 10 into the rings 16 and 17 of the ring-handled medical instruments.

In general, as seen in non-limiting FIG. 1, the rack 1 of the invention has a rack left side 2, a rack front side 3, a rack right side 4, a rack rear 5, a rack top 6, the bottom of the rack 1 (not shown) can be a flat base (adds stability to the rack in use) or may be the result of a molding or stamping of the tray construction material (and result in a hollow underside where the view from the bottom may be of any suitable contour or follows the contours of the top view). Such “hollow underside construction” has a benefit of reducing the weight of the rack, and lesser construction costs in that lesser material is needed, but results in lesser stability than a flat surfaced bottom or a rack of greater weight. The rack 1 may also be made of any suitable material such as aluminum, stainless steel, or plastic, machined and/or molded or 3-D Printed in accordance with techniques known in the art once the design of the embodiments of the present invention are known from this Specification. When plastic is used, especially when 3-D printing is used, a poly-lactic acid is the preferred (non-limiting) material for construction. Other materials for construction of the rack 1 can be selected as desired from metals that can themselves be cleaned and reused as desired or from plastics as stated above or from ceramic materials.

The benefits above at the cost of lesser stability can be balanced in varying degrees as desired with alternative bottoms such as mesh bottoms or a flat sheet of a thin, solid plate of bottom material attached to an otherwise hollowed bottom as described above (gaining the somewhat better stability of a flat bottom, and still having a lesser weight that of a completely solid rack 1. One should strive to have (but is not required to have) the rack 1 be of a such weight that the placement of the ring-handled instruments into the rack will not readily destabilize the rack into tipping over or undesirably move across the surface on which it is placed for loading such ring-handled instruments therein.

The rack 1 top 6 has a “lower ring receiving channel” 9 for a first ring-handle of a ring-handled instrument (as shown in FIG. 9). The rack 1 top has multiple top grooves 7 and corresponding multiple top groove walls 8 defining top grooves 7 to receive non-ring portions of the handle or shank 19 of the ring-handled instruments. Usually, the distal tips 18 of the instruments extend beyond the top grooves 7 at the rack 1 front side 3, but while less preferable, instruments where the distal tip 18 does not extend beyond the front side 3 of the rack 1 are perfectly suitable as well.

The lower ring receiving channel 9 extends the full distance between and through the wall of the rack 1 right side 3 and the wall of rack 1 left side 2. The channel contains alternating ring-channel grooves 20 and ring-channel groove walls 21 defining ring-channel grooves 20 so that the lower-ring of the ring-handled instrument can be seated within the ring-channel groove 20 defined by the respective groove walls 21. The rack 1 left side 2 and right side 4 are essentially mirror images of each other can be essentially of any shape as long as they contain the opening of the left and right end points of the lower ring receiving channel 9. However, they preferably are the same shape as the ring-channel groove walls 21.

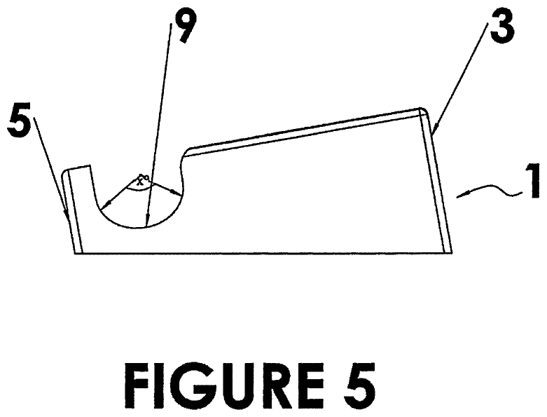

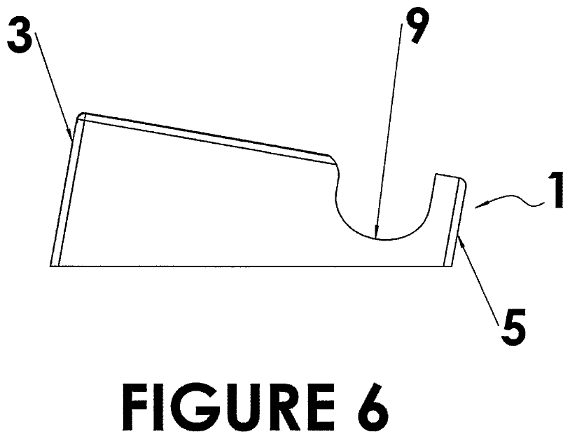

The height (relative to the bottom) of the rack 1 front wall 3 at the top groove 7 bottom 22 should be greater than the height (relative to the bottom) of the rack 1 where that same top groove 7 meets the lower ring receiving channel 9, at 23. This helps to keep the rings of the various ring-handled instruments aligned for introduction of the “U-shaped” stringers 10, by preventing slippage of some of ring-handled instruments toward the rack 1 front side 3. Although the top groove 7 bottom 22 height at tray 1 front side 3 and at position 23 can be the same, this is less desirable. This is an important distinction from racks that have a top groove for receiving instruments where the bottom of the groove at the front side 3 of the rack 1 is of less height than near the ring receiving end. In a non-limiting preferred embodiment, the angle formed between the top groove 7 bottom 22 and the rack 1 front side 3 is from 1° to 25° below the horizontal as running from the rack 1 front side toward the rack 1 rear side 5. Angles of any size in the range can be used, and more preferably are chosen from 1°, 2°, 3°, 4°, 5°, 6°, 7°, 8°, 9°, 10°, 11°, 12°, 13°, 14°, 15°,16°, 17°, 18°, 19°, 20°, 21°, 22°, 23°, 24°, and 25°, and any range constructed from any of the foregoing as long as the upper end of the range is greater than the lower end of the range. Greater angles are possible without departing from the invention but are less desirable. Although the rack 1 front side 3 and rear side 6 (as shown in FIGS. 5 and 6) are shown as being angled, they may be perfectly vertical or any desired angle. The top groove wall 8 height above the bottom of top groove 7 is shown in FIG. 1 as dimension “b” and is typically of from 0.375 cm (0.15 inches) to 1.25 cm (0.5 inches) in particular 0.375 cm (0.15 inches), 0.438 cm (0.175 inches), 0.5 cm (0.2 inches), 0.563 cm (0.225 inches), 0.625 cm (0.25 inches), 0.688 cm (0.275 inches), 0.75 cm (0.3 inches), 0.813 cm (0.325 inches), 0.875 cm (0.35 inches), 0.938 cm (0.375 inches), 1 cm (0.4 inches), 1.063 cm (0.425 inches), 1.125 cm (0.45 inches), 1.188 cm (0.475 inches), and 1.25 cm (0.5 inches) (any one of which can be a lower end or an upper end of a new range provided the upper end of such new range is greater than the lower end of such new range), and the width of top grooves 7 are shown in FIG. 1 as dimension “a” and are typically from 0.625 cm (0.25 inches) to 1 cm (0.4 inches), in particular 0.625 cm (0.25 inches), 0.688 cm (0.275 inches), 0.75 cm (0.3 inches), 0.813 cm (0.325 inches), 0.875 cm (0.35 inches), 0.938 cm (0.375 inches), 1 cm (0.4 inches) (any one of which can be a lower end or an upper end of a new range provided the upper end of such new range is greater than the lower end of such new range). Dimension “a” can be larger if desired, but should not be smaller since smaller sizes would not allow for the instrument to seat well in the groove. Dimension “b” can be of either smaller or larger sizes, but smaller top groove wall 8 heights “b” risk the instruments not being retained well in place, and overly large sizes of dimension “b” mean that instruments might seat so deeply that placement and removal of the instruments from rack 1 become cumbersome or difficult.

The shape of the lower ring receiving channel 9 is typically a circular arc at the bottom of the device being a circumferential arc subtending “x°” (see FIG. 5) of up to not more than 180° of arc centered on the bottom of the ring receiving channel 9; preferably, an arc of 120°-180°, 135°-180°, 150°-180°, 165°-180°, 170°-180°, 175°-180° or 180°, more preferably 179°-180° (although any specific number of degrees within any of the foregoing ranges, especially any particular number of degrees of at least 120° and not exceeding 180° will suffice and are acceptable as well) with the further extension of the channel continuing tangentially to the endpoints of the arc (which allows for accommodation of multiply shaped and sized ring-handled instruments). The linear dimension of widest part of the ring receiving channel 9 is generally from 3.375 cm (1.35 inches) to 3.75 cm (1.5 inches), more preferably 3.53 cm (1.41 inches) to 3.55 cm (1.42 inches), most preferably about 3.545 cm (1.418 inches). This allows for easy entry and exit most, if not all, of the ring-handled medical instruments in common use in medical procedures into the rack 1 and for easy stringing of the instruments with the “U-shaped” stringers 10 while the instruments are retained in the rack 1. Larger ring-receiving channels can be utilized for the rare situation where larger ring-handles of certain ring-handled instruments are needed. The lower ring receiving channel is interrupted with ring-channel grooves 20 and ring-channel groove walls 21, defining one another so that ring-channel grooves 20 and ring-channel groove walls 21 help keep the individual ring-handled instruments in position for the stringing with the “U-shaped” stringers 10. The height of the ring-channel groove wall 21 is measured along the radius that defines the arc of x° (see FIG. 5) and is defined as the distance that ring-channel groove wall 21 extends radially inward into the ring-channel groove 20 and is shown at one position in FIG. 1 as dimension “c”. Dimension “c” is preferably of 0.25 cm (0.1 inches) to 0.625 cm (0.25 inches), in particular 0.25 cm (0.1 inches), 0.31 cm (0.125 inches), 0.375 cm (0.15 inches), 0.438 cm (0.175 inches), 0.5 cm (0.2 inches), 0.563 cm (0.225 inches), 0.625 cm (0.25 inches) (any one of which can be a lower end or an upper end of a new range provided the upper end of such new range is greater than the lower end of such new range). The width of the ring-channel groove 20 generally varies with depth as it is curved to better accommodate the ring-handles. However, rectangular ring-channel grooves 20 are acceptable as well. At the ring-channel groove wall 21 height that is most radially inward into the ring-grooved channel along a radius defining the circumferential arc of angle x, the distance to the adjacent ring-channel groove wall 21 that define a single ring-channel groove 20, the distance “d” (shown in FIG. 1) is typically 0.625 cm (0.25 inches) to 1 cm (0.4 inches), in particular 0.625 cm (0.25 inches), 0.688 cm (0.275 inches), 0.75 cm (0.3 inches), 0.813 cm (0.325 inches), 0.875 cm (0.35 inches), 0.938 cm (0.375 inches), 1 cm (0.4 inches) (any one of which can be a lower end or an upper end of a new range provided the upper end of such new range is greater than the lower end of such new range). Larger and smaller dimensions “c” and “d” can be used but are less preferable as smaller and larger dimensions risk the instruments not being seated properly.

The rack 1 can be made of any suitable material and can be metal, plastic, or ceramic, among others as desired and known in the art. It can, for example, without limitation, be constructed from multiple parts or molded, milled, machined, stamped, or 3-D printed as is known in the art. Other techniques and materials are also possible without departing from the essence of the invention. As stated, the rack 1 can be made of metals or plastics that are known for being suitable for use in such operations. Typical suitable metals are stainless steel and anodized aluminum, among others, such as titanium. Stainless Steel is preferred.

In some embodiments the ring-handled medical instrument stringer rack 1 further has a driver tool storage channel 30 (seen in FIG. 17). The size and shape of the driver tool storage channel can be of any desired size and shape as long as the driver tools can be inserted into the channel and stored there. When the driver tools are “U-shaped” or doubly hinged, no other securing feature is required as the driver tool and be inserted into and through the channel and rotated (U-shaped driver tools) or once inserted into and through the driver tool storage channel the legs (of the doubly hinged alternative driver tool or the L-shaped tool with a further hinged element) can be rotated to secure the driver tool in place.

In other embodiments, the ring-handled medical instrument stringer rack 1 further contains one or more driver tool rest grooves (seen as 31a, 31b, and 31c in FIG. 17). In some embodiments, only one, or two, or all three of these driver rest grooves are present, and in other embodiments additional such driver rest grooves may also be present. However, preferably, when present, 1, 2, or 3 of such driver rest grooves are present. Most preferably three such grooves are present for convenience of use. The driver rest grooves 31a, 31b, and 31c (when present) extend from and through the left side 2 to and through the right side 4 at distances spaced between the front side and the lower ring receiving channel 9, but still leaving the front 3 and the lower ring receiving channel 9 edges not within the driver rest grooves (such as shown in FIG. 17). In general, the driver tool has a cross sectional diameter of about 0.15 inches to about 0.25 inches and the driver tool grooves are sized slightly larger such as from about 0.18 inches to about 0.3 inches so as to be able to seat the driver tool into the driver too rest groove. However, both larger and smaller sizes of each are within the choice of the individual without departing from the invention as long as the driver tool can seat at least partially within the driver tool rest groove. Polygonal shaped cross sections of driver tools are assumed to have cross-sectional diameters of the smallest circle that will circumscribe such polygonal cross section.

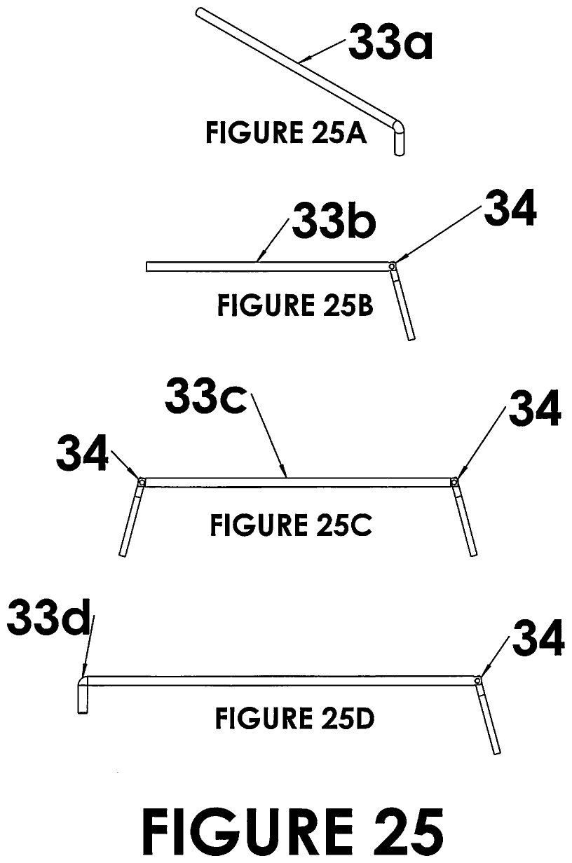

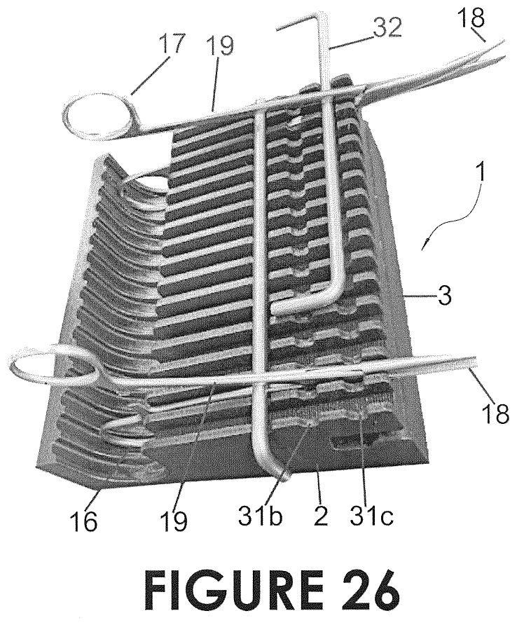

In some embodiments, the ring-handled medical instrument stringer rack 1 further has a driver tool 32 for use in connection therewith, one embodiment of the driver tool 32 is seen in FIG. 22. The driver tool may be of any desired shape, and is typically selected from a straight bar of any cross-sectional shape, may be of an “L” shape, or a “U” (curved or rectangular shape, where the rectangular shape can have rounded or sharp corners). The generally rectangular “U” shape which may also be described as a “staple” shape is seen in non-limiting FIG. 22. A fixed “L” shape driver tool is depicted as element 33a in FIG. 25A. A single hinged alternative driver tool is depicted as element 33b in FIG. 25B and as element 33d in FIG. 25D and can be used an either a simple straight bar or an “L shaped” element depending on the position of the relationship between the portions extending from the hinge. A third alternative is seen as element 33c as seen in FIG. 25C, having two spaced apart hinges 34. A yet further alternative is an “L” shaped tool having a hinged portion at the opposite end from the small leg of the “L” (see FIG. 25D). In general, the driver tool has a longer portion 35 for seating into the driver rest grooves and shorter legs 36 (see FIG. 22). For the “U-shaped” driver tool 32, the longer portion 35 is generally slightly longer than the width of the stringer rack front side 3 so that when properly seated into the driver tool rest groove (31a, 31b, or 31c) across the entire width of the stringer rack 1, the legs 36 can hug the sides of the stringer rack 1. The same applies to the double hinged alternative driver tool seen in FIG. 25C. When the alternative driver tool is a straight bar or a singly hinged one (see FIG. 25B) or an “L-shaped” tool (see FIG. 25A), the longer side of the tool can be of any reasonably convenient size but generally is slightly longer than the width of the stringer rack 1. The shorter side, when present is less than the height of the bottom of the lowest resting point of the driver tool when seated into the lowest positioned driver tool resting groove so as to allow for complete seating into the appropriate grooves.

When the driver tool 32 (or one of its alternatives as shown in FIG. 25) is used, the ring-handled medical instruments are loaded into the rack 1 and a driver in threaded over the top surface of the rack 1 between the separated portions of the shanks 19 of the ring-handled instruments. The driver is then slid toward the front 3 and seated into the most forward driver rest groove (of 31a to 31c) possible (if the grooves are present) and as far as possible toward the front 3 if the driver rest grooves are not present, each dictated by the smallest instruments that are in the rack 1. Then, if needed, a second driver tool can be threaded over the top surface of the rack 1 (at a point closer to the front 3 than the first driver tool 32 was placed) and between the separated portions of the shanks 19 of the longer ring-handled instruments and the second driver tool is then moved as much as possible toward the front surface 3 of the rack 1. If the driver rest grooves 31 (31a, 31b, and/or 31c) are present and there is a suitable unoccupied driver rest groove, the second driver tool can be seated into the unoccupied driver rest groove. Thereafter, a “U-shaped stringer” can be strung through the aligned lower rings and through the aligned upper rings and a second “U-shaped stringer” is strung in the opposite direction through the same sets of aligned ring handles.

The “U-shaped” stringers 10 are subjected to the disinfection/decontamination/sterilization processing to which the stringed instruments are to be subject and thus must be capable of withstanding the conditions of such operations. They can be made of metals that are known for being suitable for use in such operations. Typical suitable metals are stainless steel and anodized aluminum, among others. Stainless Steel is preferred. The “U-shaped” stringers, other than generally being “U-shaped” can have any configuration. In some preferred embodiments they are constructed from metal rods of any appropriate cross-section and simply bent at two points, one defining the transition from first arm 11 to the “connection” 13 between the two arms, and the other defining the transition from the “connection” 13 between the two arms and the second arm 12. In some embodiments cylindrical rods having circular cross section are suitable, in others rods having square, pentagonal, hexagonal, etc. cross-section can be used. In some embodiments rods of ovoid cross section can also be used. Alternatively, the “U-shaped” stringers may be molded without having to “bend” them into shape. Other options known in the art are also acceptable. The length of the “connection portion” can be any that is desired, but is preferably 5-10 cm (about 2-4 inches), more preferably 7-8 cm (2.8-3.2 inches), still more preferably 10 cm (4 inches) to assure that when strung, the distal tip of the instrument is in the open position and exposed for cleaning/disinfection. Advantageous, non-limiting lengths of the arms 11 and 12 are 12.5 cm (5 inches) to 30 cm (12 inches) or any convenient size depending upon the number of instruments required for a procedure and the length of the tray to which the instrument stringer is to be placed. Advantageous non-limiting cross-sectional diameters for cylindrical rods are 0.3 cm (0.12 inches) to 0.6 cm (0.24 inches) with 0.4625 cm (0.185 inches) being particularly advantageous. For non-circular regular polygon cross-sectional rods, these same dimensions can be used assuming such rods have their vertices on a circle of such diameter.

The various driver tools and alternatives can be constructed from the same set of alternative materials as the stringers 10, but the driver tools need not be the same material as the stringers 10 in any particular embodiment. Thus, stringers 10 can be made of anodized aluminum and the driver tools can be made of stainless steel or vice-versa. In addition, the driver tools may also be constructed of ceramic, plastic, or glass or other metal such as titanium. However stainless steel and anodized aluminum are more preferred.

Once the instruments requiring sterilization/disinfection are placed in the rack 1 (see FIGS. 9-11), in a first alternative, a first “U-shaped” 10 stringer (of FIGS. 7-8) is inserted through the rings of the various ring-handled instruments (while the instruments remain in the rack 1) in a first direction (the particular direction of stringing the first “U-shaped” stringer 10 through the rings is not of any concern and is entirely up to the person performing the stringing operation). Then a second “U-shaped” stringer is strung through the same ring-handled instruments in the opposite direction (the last strung instrument in the first stringing is the first strung instrument in the second stringing). Once all of the instruments in the rack have both “U-shaped” stringers strung through their respective rings, if used, the various driver tools are removed at this point, and the first free arm 11 and the second free arm 12 of each of the two so strung “U-shaped” stringers is gripped and pulled in opposite directions so that the “connections 13 between the first arm 11 and the second arm 12 of each of the two “U-shaped” stringers is pulled tightly against the group of strung instruments, which can then be transported to a cleaning/decontamination/disinfection operation. Alternatively, instead of pulling the free arms of the “U-shaped” stringers outward away from the instruments being strung, their respective “connecting portions 13 can be pushed toward the instruments being strung. The same rack and procedure can be used for a single “U-shaped” stringer with a hinged, bar closure, or other locking mechanism as described below.

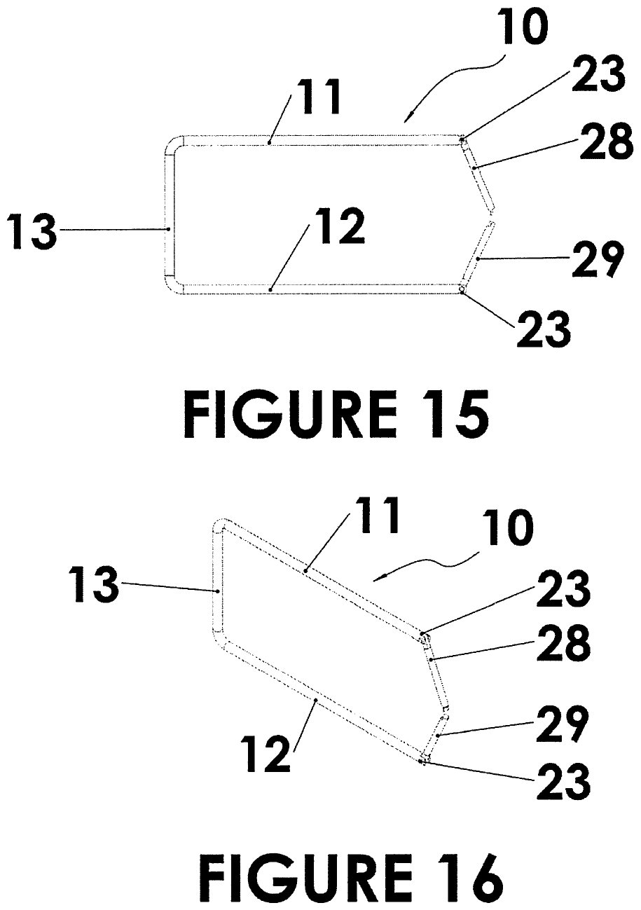

One non-limiting type of “U-shaped” stringer is shown in FIGS. 7-8, which are the basic “U-shaped” stringers. Since these have no locking mechanism, these are used in pairs as described above. A first non-limiting alternative is the stringer shown in FIGS. 13 and 14. These have the arms 11 and 12 and the connection 13 therebetween, but in addition, at the free end of arm 11 is a hinged connection 23 to which crossbar 24 is connected which further has narrowed portion 25 and end 26. In use of this first alternative, cross bar 24 is rotated so as to be a straight extension of stringer arm 11, the arms 11 and 12 are strung through the ring handles of the ring-handled instruments, cross bar 24 is then rotated so that narrowed portion 25 of cross bar 24 is received in narrowed portion receiving area 27 present in arm 12, typically using end 26 to appropriately move cross bar 24. When using this alternative “U-shaped” stringer, one only need use one stringer rather than the two described above for the simple stringer of FIGS. 5-7. In a second alternative stringer as shown in FIGS. 15 and 16, the stringer has a partial cross bar 28 and 29 attached at hinges 23, respectively, on arms 11 and 12. In use, partial cross bars 28 and 29 are rotated to be straight extensions of the respective arms to which they are attached. The ring-handled instruments are then strung on the stringer as previously described in the first alternative stringer embodiment. Then the two partial cross bars 28 and 29 are rotated to contact each other in a locking relationship. Each of the aforementioned “U-shaped” stringers (the simple type first described, as well as the two alternative versions are known in the art and commercially available.

Once the stringers have been tightened against the strung instruments, or locked as appropriate, into a group of instruments, the so strung group of instruments can be removed as a group and transported (whether in a tray designed for such purpose or directly without such a tray) to a decontamination and/or disinfection/or sterilization station for accomplishing decontamination and/or disinfection and/or sterilization of such instruments.

A first non-limiting embodiment is a ring-handled instrument rack comprising a body having a top, a bottom, and four side walls connecting the bottom to the top; said four side walls being one each of a front side wall, a left side wall, a right side wall, and a rear side wall; said top having top grooves defined by top groove walls; said top grooves extending from and through said front side wall toward said rear side wall, and terminating at a lower ring receiving channel; said lower ring receiving channel extending from and through said left side wall to and through said right side wall; said lower ring receiving channel having ring channel grooves and ring channel groove walls defining individual ring-handled instrument ring receiving areas; said ring receiving channel having a cross section when viewed from either said right side wall or said left side wall of an arc of not more than 180° and further extending tangentially from the endpoints of said arc so that said ring receiving channel groove walls meet with said top groove walls on the side of the ring receiving channel closest to said front side and said ring receiving channel grooves meet with said top grooves on the side of said ring receiving channel closest to said front side; said top grooves at the bottom thereof having an angle of 0° to 25° below the horizontal when running from said front side toward said rear side; each top groove and each ring receiving channel groove being sized to accommodate ring-handled medical instruments.

A second non-limiting embodiment is a “U-shaped” stringer for stringing ring-handled medical instruments for use in conjunction with the rack of the first non-limiting embodiment wherein the stringer is selected from the group consisting of (a) a single piece, (b) a construction of two arms each arm having a first end and a second end, and a connection portion between one of said first end of said first arm and either of said first end or said second end of said second arm, and (c) either of (a) or (b) further comprising a locking cross bar for locking the “U-shaped” stringer after said ring-handled medical instruments have been strung with said stringer whereby said ring-handled instruments remain strung onto said stringer until intentionally released therefrom, said “U-shaped” stringer connection potion forming either a rounded connection portion or a substantially flat connection portion.

A third non-limiting embodiment is a method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of the first non-limiting embodiment; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm to thread one of said first and second arms through said lower ring-shaped handle and the other of said first and second arms through said upper ring-shaped handle; each threading being while said ring-shaped medical instruments remain in said rack;
c) utilizing a second “U-shaped” stringer also having a first arm, a second arm, and a connection portion between said first arm and said second arm to thread one of said first and second arms through said lower ring-shaped handle and the other of said first and second arms through said upper ring-shaped handle; each threading being while said ring-handled medical instruments remain in said rack; said second “U-shaped” stringer beginning to string said ring-handled instruments with the last ring-handled instrument to be strung in step b) above and ending with the first ring-handled instrument to be strung in step b) above;
d) causing the free arms of said first “U-shaped” stringer to be drawn away from the free arms of said second “U-shaped” stringer, resulting in the first “U-shaped” stringer connection portion to be drawn toward the second “U-shaped” stringer connection portion, thereby clamping the stringers against the group of so strung ring-handled instruments; and
e) transporting the clamped, strung instruments to at least one of an assembly operation, a decontamination operation, and/or a disinfection operation;
wherein either or both of said “U-shaped” stringers have stringer connection potions forming either a rounded connection portion or a substantially flat connection portion.

A fourth non-limiting embodiment is a method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of the first non-limiting embodiment; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm, a hinged cross bar extending from one of said first arm and said second arm, and a cross bar receiving area on the other of said first arm and said second arm, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion; and
c) causing the crossbar to rotate so that the distal end of said cross bar seats into said cross bar receiving portion and locks in place.; and
e) transporting the clamped, strung instruments to at least one of an assembly operation, a decontamination operation, and/or a disinfection operation.

A fifth non-limiting embodiment is a method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of the first non-limiting embodiment; said ring-handled medical instruments having (I) a pair of ring shaped handles, one of which being designated a lower ring-shaped handle and a second of which being designated an upper ring-shaped handle and (II) a distal tip;
b) utilizing a “U-shaped” stringer, having a first arm, a second arm, and a connection portion between said first arm and said second arm, a hinged partial cross bar extending from each of said first arm and said second arm, each of said partial cross bars having a locking end distal to the hinge such that on rotation of said partial cross bars toward each other, said locking ends engage with each other in locking relationship, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
c) causing the partial cross bars to rotate so that the distal ends of the partial cross bars engage each other in a locking relationship; and
e) transporting the strung instruments to at least one of an assembly operation, a decontamination operation, and/or a disinfection operation.

A sixth noon-limiting embodiment is a ring-handled instrument rack of the first non-limiting embodiment for use in preparation for a procedure selected from at least one of an assembly operation, an assembly count operation, a storage procedure, a decontamination operation, a sterilization process, and/or a disinfection operation, involving such instruments, which preparation comprises
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm to thread one of said first and second arms through said lower ring-shaped handle and the other of said first and second arms through said upper ring-shaped handle; each threading being while said ring-shaped medical instruments remain in said rack;
c) utilizing a second “U-shaped” stringer also having a first arm, a second arm, and a connection portion between said first arm and said second arm to thread one of said first and second arms through said lower ring-shaped handle and the other of said first and second arms through said upper ring-shaped handle; each threading being while said ring-handled medical instruments remain in said rack; said second “U-shaped” stringer beginning to string said ring-handled instruments with the last ring-handled instrument to be strung in step b) above and ending with the first ring-handled instrument to be strung in step b) above; and
d) causing the free arms of said first “U-shaped” stringer to be drawn away from the free arms of said second “U-shaped” stringer, resulting in the first “U-shaped” stringer connection portion to be drawn toward the second “U-shaped” stringer connection portion, thereby clamping the stringers against the group of so strung ring-handled instruments wherein either or both of said “U-shaped” stringer have connection portions that form rounded or substantially flat connection portions.

A seventh non-liming embodiment is the ring-handled instrument rack of the first non-limiting embodiment for use in preparation for a procedure selected from at least one of an assembly operation, an assembly count operation, a storage procedure, a decontamination operation, a sterilization process, and/or a disinfection operation, involving such instruments, which preparation comprises
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm, a hinged cross bar extending from one of said first arm and said second arm, and a cross bar receiving area on the other of said first arm and said second arm, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
c) causing the crossbar to rotate so that the distal end of said cross bar seats into said cross bar receiving portion and locks in place.

An eighth non-limiting embodiment is the ring-handled instrument rack of the first non-limiting embodiment for use in preparation for a procedure selected from at least one of an assembly operation, an assembly count operation, a storage procedure, a decontamination operation, a sterilization process, and/or a disinfection operation, involving such instruments, which preparation comprises
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a “U-shaped” stringer, having a first arm, a second arm, and a connection portion between said first arm and said second arm, a hinged partial cross bar extending from each of said first arm and said second arm, each of said partial cross bars having a locking end distal to the hinge such that on rotation of said partial cross bars toward each other, said locking ends engage with each other in locking relationship, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
c) causing the partial cross bars to rotate so that the distal ends of the partial cross bars engage each other in a locking relationship.

A ninth non-limiting embodiment is the ring-handled instrument rack of the first non-limiting embodiment further comprising from at least one driver tool rest groove, said driver tool rest groove being along the top surface of said rack, each said driver tool rest groove being a groove between and through each of said right side of said rack and said left side of said rack, being arcuate in shape and located between said front of said rack and closer to said front of said rack than said lower ring receiving channel.

A tenth non-limiting embodiment is the ring-handled instrument rack of the ninth non-limiting embodiment further comprising a driver tool storage channel, said channel being a void between and through said left side wall and said right side wall located below the top surface of said driver tool storage channel.

An eleventh non-limiting embodiment is a method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of the ninth non-limiting embodiment; said ring-handled medical instruments having (I) a pair of ring shaped handles, one of which being designated a lower ring-shaped handle and a second of which being designated an upper ring-shaped handle, (II) a distal tip, (III) a pivot point, and (IV) an upper shank portion and a lower shank portion, the upper shank portion connecting said upper ring to said pivot point and said lower shank portion connecting said lower ring to said pivot point;
b) introducing a driver tool over the top of said rack, between the upper shank portion and the lower shank portion of said respective ring handled medical instruments and moving said driver tool toward said front of said rack as far as possible and optionally seating said driver tool into said driver tool rest channel;
c) utilizing a first “U-shaped” stringer, having a first arm, a second arm, and a connection portion between said first arm and said second arm to thread one of said first and second arms through said lower ring-shaped handle and the other of said first and second arms through said upper ring-shaped handle; each threading being while said ring-shaped medical instruments remain in said rack, and optionally removing said driver tool;
d) utilizing a second “U-shaped” stringer also having a first arm, a second arm, and a connection portion between said first arm and said second arm to thread one of said first and second arms through said lower ring-shaped handle and the other of said first and second arms through said upper ring-shaped handle; each threading being while said ring-handled medical instruments remain in said rack; said second “U-shaped” stringer beginning to string said ring-handled instruments with the last ring-handled instrument to be strung in step c) above and ending with the first ring-handled instrument to be strung in step c) above and optionally remove said driver tool if not previously removed;
e) causing the free arms of said first “U-shaped” stringer to be drawn away from the free arms of said second “U-shaped” stringer, resulting in the first “U-shaped” stringer connection portion to be drawn toward the second “U-shaped” stringer connection portion, thereby clamping the stringers against the group of so strung ring-handled instruments, and if sid driver tool has not previously been removed, removing said driver tool; and
f) transporting the clamped, strung instruments to at least one of an assembly operation, a decontamination operation, and/or a disinfection operation; wherein either or both of said “U-shaped” stringers have stringer connection potions forming either a rounded connection portion or a substantially flat connection portion.

A twelfth non-limiting embodiment is a method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of the ninth non-limiting embodiment; said ring-handled medical instruments having (I) a pair of ring shaped handles, one of which being designated a lower ring-shaped handle and a second of which being designated an upper ring-shaped handle, (II) a distal tip, (III) a pivot point, and (IV) an upper shank portion and a lower shank portion, the upper shank portion connecting said upper ring to said pivot point and said lower shank portion connecting said lower ring to said pivot point;
b) introducing a driver tool over the top of said rack, between the upper shank portion and the lower shank portion of said respective ring handled medical instruments and moving said driver tool toward said front of said rack as far as possible and optionally seating said driver tool into said driver tool rest channel;
c) utilizing a first “U-shaped” stringer, having a first arm, a second arm, and a connection portion between said first arm and said second arm, a hinged cross bar extending from one of said first arm and said second arm, and a cross bar receiving area on the other of said first arm and said second arm, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion; and
d) causing the crossbar to rotate so that the distal end of said cross bar seats into said cross bar receiving portion and locks in place; and
e) transporting the clamped, strung instruments to at least one of an assembly operation, a decontamination operation, and/or a disinfection operation.

A thirteenth non-limiting embodiment is a method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of the ninth non-limiting embodiment; said ring-handled medical instruments having (I) a pair of ring shaped handles, one of which being designated a lower ring-shaped handle and a second of which being designated an upper ring-shaped handle, (II) a distal tip, (III) a pivot point, and (IV) an upper shank portion and a lower shank portion, the upper shank portion connecting said upper ring to said pivot point and said lower shank portion connecting said lower ring to said pivot point;
b) introducing a driver tool over the top of said rack, between the upper shank portion and the lower shank portion of said respective ring handled medical instruments and moving said driver tool toward said front of said rack as far as possible and optionally seating said driver tool into said driver tool rest channel;
c) utilizing a “U-shaped” stringer, having a first arm, a second arm, and a connection portion between said first arm and said second arm, a hinged partial cross bar extending from each of said first arm and said second arm, each of said partial cross bars having a locking end distal to the hinge such that on rotation of said partial cross bars toward each other, said locking ends engage with each other in locking relationship, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
d) causing the partial cross bars to rotate so that the distal ends of the partial cross bars engage each other in a locking relationship; and
e) removing said driver tool;
f) transporting the strung instruments to at least one of an assembly operation, a decontamination operation, and/or a disinfection operation.

I/we claim:

1. A ring-handled instrument rack comprising a body having a top, a bottom, and four side walls connecting the bottom to the top; said four side walls being one each of a front side wall, a left side wall, a right side wall, and a rear side wall; said top having top grooves defined by top groove walls; said top grooves extending from and through said front side wall toward said rear side wall, and terminating at a lower ring receiving channel; said lower ring receiving channel extending from and through said left side wall to and through said right side wall; said lower ring receiving channel having ring channel grooves and ring channel groove walls defining individual ring-handled instrument ring receiving areas; said ring receiving channel having a cross section when viewed from either said right side wall or said left side wall of an arc of not more than 180° and further extending tangentially from the endpoints of said arc so that said ring receiving channel groove walls meet with said top groove walls on the side of the ring receiving channel closest to said front side and said ring receiving channel grooves meet with said top grooves on the side of said ring receiving channel closest to said front side; said top grooves at the bottom thereof having an angle of 0° to 25° below the horizontal when running from said front side toward said rear side; each top groove and each ring receiving channel groove being sized to accommodate ring-handled medical instruments.

2. A “U-shaped” stringer for stringing ring-handled medical instruments for use in conjunction with the rack of claim 1 wherein the stringer is selected from the group consisting of (a) a single piece, (b) a construction of two arms each arm having a first end and a second end, and a connection portion between one of said first end of said first arm and either of said first end or said second end of said second arm, and (c) either of (a) or (b) further comprising a locking cross bar for locking the “U-shaped” stringer after said ring-handled medical instruments have been strung with said stringer whereby said ring-handled instruments remain strung onto said stringer until intentionally released therefrom, said “U-shaped” stringer connection potion forming either a rounded connection portion or a substantially flat connection portion.

3. A method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm to thread one of said first and second arms through said lower ring-shaped handle and the other of said first and second arms through said upper ring-shaped handle; each threading being while said ring-shaped medical instruments remain in said rack;
c) utilizing a second “U-shaped” stringer also having a first arm, a second arm, and a connection portion between said first arm and said second arm to thread one of said first and second arms through said lower ring-shaped handle and the other of said first and second arms through said upper ring-shaped handle; each threading being while said ring-handled medical instruments remain in said rack; said second “U-shaped” stringer beginning to string said ring-handled instruments with the last ring-handled instrument to be strung in step b) above and ending with the first ring-handled instrument to be strung in step b) above;
d) causing the free arms of said first “U-shaped” stringer to be drawn away from the free arms of said second “U-shaped” stringer, resulting in the first “U-shaped” stringer connection portion to be drawn toward the second “U-shaped” stringer connection portion, thereby clamping the stringers against the group of so strung ring-handled instruments; and
e) transporting the clamped, strung instruments to at least one of an assembly operation, a decontamination operation, and/or a disinfection operation;
wherein either or both of said “U-shaped” stringers have stringer connection potions forming either a rounded connection portion or a substantially flat connection portion.

4. A method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm, a hinged cross bar extending from one of said first arm and said second arm, and a cross bar receiving area on the other of said first arm and said second arm, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion; and
c) causing the crossbar to rotate so that the distal end of said cross bar seats into said cross bar receiving portion and locks in place; and
e) transporting the clamped, strung instruments to at least one of an assembly operation, a decontamination operation, and/or a disinfection operation.

5. A method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a “U-shaped” stringer, having a first arm, a second arm, and a connection portion between said first arm and said second arm, a hinged partial cross bar extending from each of said first arm and said second arm, each of said partial cross bars having a locking end distal to the hinge such that on rotation of said partial cross bars toward each other, said locking ends engage with each other in locking relationship, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
c) causing the partial cross bars to rotate so that the distal ends of the partial cross bars engage each other in a locking relationship; and
e) transporting the strung instruments to at least one of an assembly operation, a decontamination operation, and/or a disinfection operation.

6. The ring-handled instrument rack of claim 1 for use in preparation for a procedure selected from at least one of an assembly operation, an assembly count operation, a storage procedure, a decontamination operation, a sterilization process, and/or a disinfection operation, involving such instruments, which preparation comprises
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm to thread one of said first and second arms through said lower ring-shaped handle and the other of said first and second arms through said upper ring-shaped handle; each threading being while said ring-shaped medical instruments remain in said rack;
c) utilizing a second “U-shaped” stringer also having a first arm, a second arm, and a connection portion between said first arm and said second arm to thread one of said first and second arms through said lower ring-shaped handle and the other of said first and second arms through said upper ring-shaped handle; each threading being while said ring-handled medical instruments remain in said rack; said second “U-shaped” stringer beginning to string said ring-handled instruments with the last ring-handled instrument to be strung in step b) above and ending with the first ring-handled instrument to be strung in step b) above; and
d) causing the free arms of said first “U-shaped” stringer to be drawn away from the free arms of said second “U-shaped” stringer, resulting in the first “U-shaped” stringer connection portion to be drawn toward the second “U-shaped” stringer connection portion, thereby clamping the stringers against the group of so strung ring-handled instruments wherein either or both of said “U-shaped” stringer have connection portions that form rounded or substantially flat connection portions.

7. The ring-handled instrument rack of claim 1 for use in preparation for a procedure selected from at least one of an assembly operation, an assembly count operation, a storage procedure, a decontamination operation, a sterilization process, and/or a disinfection operation, involving such instruments, which preparation comprises
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a first “U-shaped” stringer, having a first arm, a second arm, and a connection portion between said first arm and said second arm, a hinged cross bar extending from one of said first arm and said second arm, and a cross bar receiving area on the other of said first arm and said second arm, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
c) causing the crossbar to rotate so that the distal end of said cross bar seats into said cross bar receiving portion and locks in place.

8. The ring-handled instrument rack of claim 1 for use in preparation for a procedure selected from at least one of an assembly operation, an assembly count operation, a storage procedure, a decontamination operation, a sterilization process, and/or a disinfection operation, involving such instruments, which preparation comprises
a) placing ring-handled medical instruments in need of at least one of a decontamination operation, and/or a disinfection operation, into a rack of claim 1; said ring-handled medical instruments having (I) a pair of ring shaped handles, one of which being designated a lower ring-shaped handle and a second of which being designated an upper ring-shaped handle and (II) a distal tip;
b) utilizing a “U-shaped” stringer, having a first arm, a second arm, and a connection portion between said first arm and said second arm, a hinged partial cross bar extending from each of said first arm and said second arm, each of said partial cross bars having a locking end distal to the hinge such that on rotation of said partial cross bars toward each other, said locking ends engage with each other in locking relationship, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
c) causing the partial cross bars to rotate so that the distal ends of the partial cross bars engage each other in a locking relationship.

9. The ring-handled instrument rack of claim 1 further comprising from at least one driver tool rest groove, said driver tool rest groove being along the top surface of said rack, each said driver tool rest groove being a groove between and through each of said right side of said rack and said left side of said rack, being arcuate in shape and located between said front of said rack and closer to said front of said rack than said lower ring receiving channel.

10. The ring-handled instrument rack of claim 1 further comprising a driver tool storage channel, said channel being a void between and through said left side wall and said right side wall located below the top surface of said driver tool storage channel.

11. A method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of claim 9; said ring-handled medical instruments having (I) a pair of ring shaped handles, one of which being designated a lower ring-shaped handle and a second of which being designated an upper ring-shaped handle, (II) a distal tip, (III) a pivot point, and (IV) an upper shank portion and a lower shank portion, the upper shank portion connecting said upper ring to said pivot point and said lower shank portion connecting said lower ring to said pivot point;
b) introducing a driver tool over the top of said rack, between the upper shank portion and the lower shank portion of said respective ring handled medical instruments and moving said driver tool toward said front of said rack as far as possible and optionally seating said driver tool into said driver tool rest channel;
c) utilizing a first “U-shaped” stringer, having a first arm, a second arm, and a connection portion between said first arm and said second arm to thread one of said first and second arms through said lower ring-shaped handle and the other of said first and second arms through said upper ring-shaped handle; each threading being while said ring-shaped medical instruments remain in said rack, and optionally removing said driver tool;
d) utilizing a second “U-shaped” stringer also having a first arm, a second arm, and a connection portion between said first arm and said second arm to thread one of said first and second arms through said lower ring-shaped handle and the other of said first and second arms through said upper ring-shaped handle; each threading being while said ring-handled medical instruments remain in said rack; said second “U-shaped” stringer beginning to string said ring-handled instruments with the last ring-handled instrument to be strung in step c) above and ending with the first ring-handled instrument to be strung in step c) above and optionally remove said driver tool if not previously removed;
e) causing the free arms of said first “U-shaped” stringer to be drawn away from the free arms of said second “U-shaped” stringer, resulting in the first “U-shaped” stringer connection portion to be drawn toward the second “U-shaped” stringer connection portion, thereby clamping the stringers against the group of so strung ring-handled instruments, and if sid driver tool has not previously been removed, removing said driver tool; and
f) transporting the clamped, strung instruments to at least one of an assembly operation, a decontamination operation, and/or a disinfection operation;
wherein either or both of said “U-shaped” stringers have stringer connection potions forming either a rounded connection portion or a substantially flat connection portion.

12. A method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of claim 9; said ring-handled medical instruments having (I) a pair of ring shaped handles, one of which being designated a lower ring-shaped handle and a second of which being designated an upper ring-shaped handle, (II) a distal tip, (III) a pivot point, and (IV) an upper shank portion and a lower shank portion, the upper shank portion connecting said upper ring to said pivot point and said lower shank portion connecting said lower ring to said pivot point;
b) introducing a driver tool over the top of said rack, between the upper shank portion and the lower shank portion of said respective ring handled medical instruments and moving said driver tool toward said front of said rack as far as possible and optionally seating said driver tool into said driver tool rest channel;
c) utilizing a first “U-shaped” stringer, having a first arm, a second arm, and a connection portion between said first arm and said second arm, a hinged cross bar extending from one of said first arm and said second arm, and a cross bar receiving area on the other of said first arm and said second arm, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion; and
d) causing the crossbar to rotate so that the distal end of said cross bar seats into said cross bar receiving portion and locks in place.; and
e) transporting the clamped, strung instruments to at least one of an assembly operation, a decontamination operation, and/or a disinfection operation.

13. A method of stringing ring-handled medical instruments in preparation for at least one of an assembly operation, an assembly count operation, a storage procedure, a decontamination operation, sterilization process, and/or a disinfection operation, comprising:
a) placing ring-handled medical instruments in need of at least one of a decontamination operation, and/or a disinfection operation, into a rack of claim 9; said ring-handled medical instruments having (I) a pair of ring shaped handles, one of which being designated a lower ring-shaped handle and a second of which being designated an upper ring-shaped handle, (II) a distal tip, (III) a pivot point, and (IV) an upper shank portion and a lower shank portion, the upper shank portion connecting said upper ring to said pivot point and said lower shank portion connecting said lower ring to said pivot point;
b) introducing a driver tool over the top of said rack, between the upper shank portion and the lower shank portion of said respective ring handled medical instruments and moving said driver tool toward said front of said rack as far as possible and optionally seating said driver tool into said driver tool rest channel;
c) utilizing a “U-shaped” stringer, having a first arm, a second arm, and a connection portion between said first arm and said second arm, a hinged partial cross bar extending from each of said first arm and said second arm, each of said partial cross bars having a locking end distal to the hinge such that on rotation of said partial cross bars toward each other, said locking ends engage with each other in locking relationship, to thread one of said first and second arms through said lower ring-shaped handle and the other of said first and second arms through said upper ring-shaped handle; each threading being while said ring-shaped medical instruments remain in said rack; said “U-shaped” stringer connection potion forming either a rounded connection portion or a substantially flat connection portion and
d) causing the partial cross bars to rotate so that the distal ends of the partial cross bars engage each other in a locking relationship; and
e) removing said driver tool;
f) transporting the strung instruments to at least one of an assembly operation, a decontamination operation, and/or a disinfection operation.
